(19) United States
(12) Patent Application Publication
(10) Pub. No.: US 2005/0113869 A1
(43) Pub. Date: May 26, 2005

(54) Armed suture with adhesively attached surgical needle

(75) Inventor: John J. Price, Gainesville, GA (US)

Correspondence Address: Ralph W. Selitto, Jr.;McCarter & English, LLP, Four Gateway Center, 100 Mulberry Street, Newark, NJ, 07102, US

(73) Assignee: Ethicon, Inc.

(21) Appl. No.: 10/722,945

(22) Filed: Nov. 26, 2003

(51) Int. Cl. A61B 17/06

(52) U.S. Cl. 606/225; 606/224; 606/223; 606/222; 606/226

(57) ABSTRACT

An adhesively attached armed suture has a needle with a suture hole formed therein. The suture has an end thereof inserted into the suture hole and is adhesively bonded therein. The adhesive has a viscosity when uncured permitting the suture to be inserted into the suture hole, and preferably has a low viscosity to facilitate easy insertion. A UV-curing adhesive may be of low viscosity and be rapidly cured to more rapidly attach the suture and needle. A “bottle-shaped” suture hole may be utilized to enhance the interlocking between the adhesive and the needle.

### FIELD OF THE INVENTION

The present invention relates to armed sutures having surgical needles attached thereto and more particularly to such armed sutures wherein the needle is attached by an adhesive.

### BACKGROUND OF THE INVENTION

The attachment of surgical needles to suture with adhesives has been tried in the past. For example, U.S. Pat. No. 3,799,169 to Beroff et al. and assigned to the present Assignee describes use of epoxy resin to bond a needle to a suture. When used with needles having a blind suture hole, epoxy is viscous, impeding the insertion of suture into the suture hole. Notwithstanding the prior art, it remains an objective to prepare armed sutures using adhesive attachment of the needle(s) which is easier to accomplish and provides greater pull strength and efficiency of production.

### SUMMARY OF THE INVENTION

The limitations of prior art, adhesively-attached, armed sutures are addressed by the present invention, which includes an armed suture having a needle with a suture hole formed therein. A suture has an end thereof inserted into the suture hole. An adhesive bonds the suture and the needle within the suture hole. The adhesive has a viscosity when uncured permitting the suture to be inserted into the suture hole.

### BRIEF DESCRIPTION OF THE FIGURES

FIG. 1 is cross-sectional view of a surgical needle at an end to which suture is attached;

FIG. 2 is a cross-sectional view like FIG. 1, but with a reduced suture hole depth and coined end;

FIG. 3 is a cross-sectional view of the needle of FIG. 1 but including a suture and adhesive material therein;

FIG. 4 is a cross-sectional view of the needle of FIG. 2 but including a suture and adhesive material therein;

FIGS. 5aa-5e are diagrammatic cross-sectional views of procedural steps conducted in accordance with the present invention for adhesively attaching a suture to a surgical needle;

FIG. 6 is a cross-sectional view like FIG. 1, but of a needle configured in accordance with an alternative embodiment of the present invention; and

FIG. 7 is a cross-sectional view like FIG. 1, but of a needle configured in accordance with another alternative embodiment of the present invention.

### DETAILED DESCRIPTION OF THE INVENTION

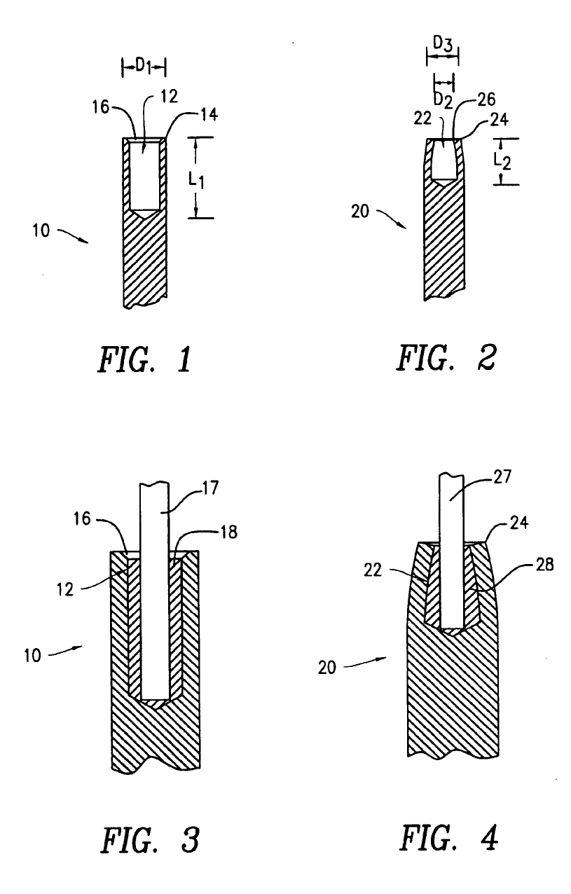

FIG. 1 shows a surgical needle 10 with a suture hole 12 formed in an end 14 thereof by means of mechanical or laser drilling. The suture hole 12 has a chamfered upper edge 16. As further described below, the configuration of the needle 10 shown in FIG. 1 is conventional except for the dimensions of the suture hole 12, viz., length L and diameter D, relative to the length and gauge of the needle. In general, there are a few competing considerations as to suture hole 12 dimensions, viz., the larger the suture hole 12, the greater the potential surface area to which as adhesive in accordance with the present invention may bond. Furthermore, the use of adhesives permits a larger suture hole 12, which can accommodate a range of suture sizes therein. From the stand point of drill wear (associated with drilling the suture hole 12) however, it is desirable for the depth of the suture hole 12 to be kept to a minimum.

FIG. 2 shows a surgical needle 20 with a suture hole 22 formed in an end 24 thereof. Relative to the suture hole 12 of FIG. 1, the suture hole 22 of FIG. 2 has a reduced length L2 and an unchamfered upper edge 26. The upper edge 26 is “coined” or swaged inwardly to form a “bottle-shaped” suture hole 22 having a reduced upper diameter D2 and a larger, lower diameter D3. This swaging effect may be accomplished by a Torrington Swaging unit obtained from Torrington Swager and Vaill End Machinery, Inc. of Waterbury, Conn.

FIG. 3 shows the needle 10 of FIG. 1 with a suture 17 inserted into the suture hole 12 and retained there by adhesive 18. The adhesive 18 preferably has a low-viscosity to allow easy insertion of the suture 17 into the suture hole 12, i.e., by readily flowing between the suture 17 and the suture hole 12 as the suture 17 is inserted into the suture hole 12. A suitable adhesive is LOCTITE ® Product 4302, which is a low viscosity, two-part, UV-curable cyanoacrylate adhesive. It has a rapid UV or visible light cure with a cyanoacrylate secondary cure mechanism. The use of adhesive 18 to attach the needle 10 to the suture 17 permits a variety of relative sizing of the needle 10 and suture 17 so joined. More particularly, the common mechanical method of swaging needles to sutures, i.e., without use of adhesives, requires close tolerances between the suture and needle. As a result only one size (diameter) suture made from a particular material may be used with only one size (diameter) suture hole. Since the adhesive 18 of the present invention bridges the gap between and bonds the suture 17 to the needle 10 (at the suture hole 12) a range of suture diameters and materials may be used with a single needle type (having a single set of suture hole 12 dimensions. This represents a significant advantage in that the number of different needles that must be manufactured, catalogued, maintained in inventory, processed, etc. may be greatly reduced, resulting in greater efficiency and substantial savings. From the standpoint of needle manufacturing, since the suture hole 12 may have a range of sizes and still be suitable for adhesive attachment, the needle 10 manufacturing costs may be reduced. For example, there may be a reduction in the number of different sized drilling apparatus used to produce the suture hole 12 in the needles 10. In addition, the drills (bits and apparatus) may be used longer because the manufacturing tolerances are not as close, so there is a reduction in wastage, testing, change outs and adjustments in needle drilling machines. Because the number of different needles is reduced, lot sizes can be increased, resulting in greater production with less setup. Because the suture hole 12 diameter can be larger than the suture 17, it is easier to insert the suture 17 into the suture hole 12. This advantage would be expressed as a reduced “hole centering” requirement, which would be particularly important in an automated process. Because the needle 10 and suture 17 are attached by adhesive, no annealing step is required to reduce the stresses caused by swaging.

FIG. 4 shows a suture 27 inserted into suture hole 22 and affixed there by adhesive 28, which may be the same type of adhesive 18 as described above in reference to FIG. 3. The coined upper edge 24 creates a bottle-shaped suture hole 22, such that when the suture hole 22 is filled with adhesive 28, the adhesive 28 also cures to a bottle-shape. This bottle-shaped adhesive 28 is mechanically locked into the suture hole 22. The mechanical interlock exhibited between the suture hole 22 and adhesive 28 allows the length of the hole 22 to be reduced while maintaining the same “pull strength,” which is the tensile strength of the conjunction of the needle 20 with the suture 27. (When the pull strength is exceeded, the suture 27 would pull out of the suture hole 22, such as during destructive testing.)

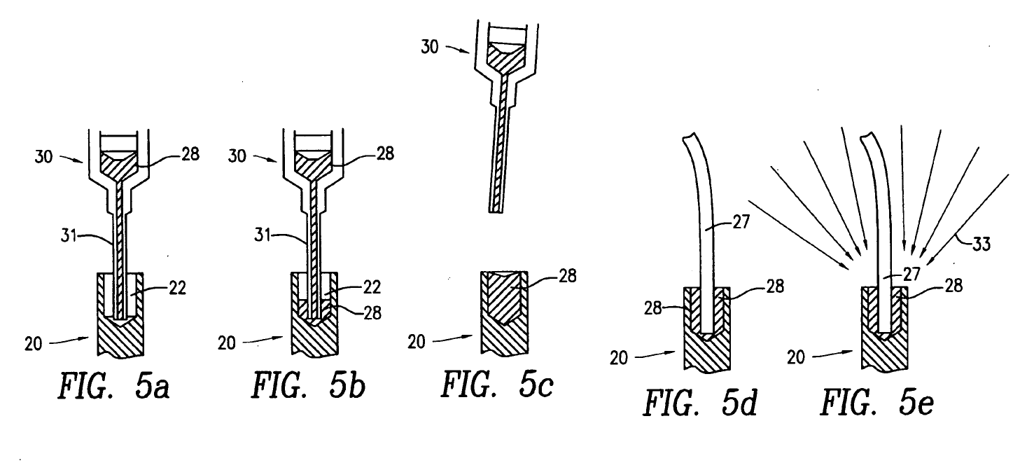

FIGS. 5a through 5e illustrate an exemplary procedure in accordance with the present invention. In FIG. 5a, a syringe 30 charged with adhesive 28 has a needle portion 31 that is inserted into the suture hole 22 of a needle 20. FIG. 5b shows the adhesive 28 being injected into the suture hole 22. The use of a syringe 30 or other graduated filling apparatus, such as a pipette, permits the controlled introduction of a suitable amount of adhesive 28 into the suture hole 22. It is desirable for the adhesive 28 injected into the suture hole 22 not to exceed an amount which would completely fill the suture hole 22 after the suture 27 is inserted. That is, adhesive 28 overflow should be avoided. It is not necessary for the adhesive 28 to completely fill the suture hole 22 when the suture 27 is inserted, in that a lesser amount of adhesive 28 may provide sufficient attachment strength for a given application.

After the predetermined amount of adhesive 28 is introduced into the suture hole 22, the syringe 30 is withdrawn, as shown in FIG. 5c. The suture 27 is then inserted into the adhesive 28 deposited in the suture hole 22, as shown in FIG. 5d. FIG. 5e illustrates the irradiation of the adhesive 28 with UV, visible light or other electromagnetic radiation 33 to aid in the curing of the adhesive 28. Depending upon the adhesive 28 used, radiation may not be required for the curing of the adhesive 28. The advantages of UV-curing adhesive 28 is that it may be dispensed in the non-viscous, liquid state to facilitate filling the suture hole 22. The adhesive 28 may then be partially or completely cured while the suture 27 is held in the adhesive 28, accomplishing attachment and allowing the attached suture 27 and needle 20 to be released by the holder. The foregoing process may be accomplished entirely manually or be aided by fixtures which, e.g., hold the needle 20 for filing, hold the syringe 30 during the injection of adhesive into the suture hole 22 and hold the suture 27 and needle 20 while the adhesive 28 is irradiated. In addition, the process may be mechanized, such that all the foregoing steps are accomplished by machines, robotically and automatically.

Instead of pre-filling the suture hole 22 with adhesive 28, the foregoing process may be varied by dipping the suture 22 into a reservoir of adhesive 28 and then inserting the adhesive-wetted end of the suture 27 into the suture hole 22. When using low viscosity adhesives, the adhesive 28 into which the suture 27 is dipped may be partially cured to increase viscosity and slow the flow of adhesive down the suture 27.

As a further alternative, either of the foregoing processes, viz. pre-filling and dipping, may incorporate the use of a catalyst. For example, the LOCTITE® 4302 adhesive mentioned above utilizes cyanoacrylate as a secondary cure mechanism. Accordingly, the suture hole 22 can be coated with cyanoacrylate prior to filling with adhesive 28 in the pre-filling approach or, prior to insertion of the dipped suture, in the dipping approach. Further, the suture 27 can be dipped, painted or sprayed with the chemical curative, e.g. cyanoacrylate, prior to being dipped, or inserted in the adhesive 28.

Curing time (and pull-out strength) is effected by the intensity and duration of exposure to radiation, to the length of time between radiation curing and testing, by the use of a chemical curative, e.g. cyanoacrylate, and other factors, e.g., heat. In some instances, a first type of curing, e.g. a short exposure to visible or UV light may partially cure the adhesive, (flash it over) followed by a more complete cure attributable to time, chemical curatives, atmospheric moisture, and/or heat and other forms of radiation (microwave). It has been observed that sterilization of armed sutures in accordance with the present invention by heat and/or irradiation contributes to curing.

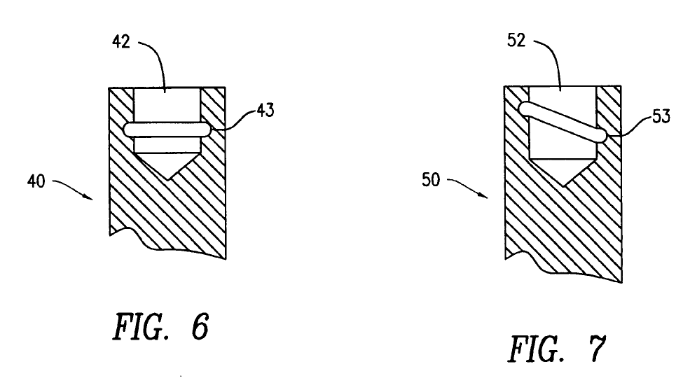

FIGS. 6 and 7 illustrate needles 40 and 50, respectively, which include grooves 43 and 53, respectively, to increase the mechanical interlocking of the adhesive 28 and the needles, 40, 50. It has been observed that increased surface roughness in the area of the suture hole 12, 22, 42, 52 increases pull-out strength. This is evident when comparing the pull-out strength of finished vs. unfinished needles. Accordingly, methods for isolating the suture hole, e.g.; 12 from finishing, such as by using water or wax plugs are advantageous. As another alternative, the suture hole 12 of a finished needle can be roughened by acid etching, by mechanical processes, such as by reaming with a rough reamer, or by laser drilling. The foregoing features of the present invention are further exemplified and illustrated by the following examples:

### EXAMPLE 1

Size 3-0 Ethilon and size 3-0 coated Vicryl sutures were used in this example. Both suture types were attached to unfinished 493962 needles, which have an nominal hole diameter of 14.5 mil. The 3-0 Vicryl was also attached to finished 522617 needles, which have a nominal hole diameter of 15.8 mil. The adhesive was applied using the suture dip method. The sample sizes varied for each set of pulls.

Each of the attachment types was pulled in varying sample sizes at less than 10 minutes from exposure and then at greater than 24 hours from exposure. The exposure times (to UV light) varied from 5 to 9 seconds.

The pull test averages were as follows:

| | Within 10 Min. of | 24 Hours from |
|---|---|---|
| Suture - Needle Combination: | Cure | Cure |
| 3-0 Ethilon - Unfinished 493962 | 0.64 lb | 0.52 lb |
| 3-0 Vicryl - Unfinished 493962 | 3.20 lb | 4.90 lb |
| 3-0 Vicryl - Finished 522617 | 0.60 lb | 1.83 lb |

This test confirmed the secondary curing effects of the LOCTITE 4302 adhesive with regard to the Vicryl suture.

### EXAMPLE 2

Sizes 4-0, 2-0 and 1 coated Vicryl were used in this study. All three suture sizes were attached to 483947 needles, which have a nominal hole diameter of 24.8 mil. The cure time for sizes 4-0 and 2-0 was 1 second, and for size 1 the cure time was 3 seconds. All attachments were performed using the syringe injection method of adhesive application.

For each suture size, n=12 samples were pulled immediately after curing, another n=12 were pulled after 30 minutes, and another n=12 were pulled after 24 hours.

All three suture sizes showed comparable initial cure strengths, as shown below. Both the 4-0 and 2-0 attachments showed steady increases in strength over time, and achieved final pull strengths which met current in-process manufacturing standards for that suture size.

| Vicryl | Exposure | Immediate | 30 Min. from | 24 hr. from |
|---|---|---|---|---|
| Suture Size | Time | Pull | cure | cure |
| 4-0 | 1 sec. | 0.71 lb | 2.17 lb | 3.07 lb |
| 2-0 | 1 sec. | 0.33 lb | 2.17 lb | 4.19 lb |
| 1 | 3 sec. | 0.54 lb | 5.28 lb | 5.14 lb |

This example showed that the adhesive 4302 from LOCTITE does in fact achieve an improved attachment strength over time when used in needle/suture attachment. It also showed that this adhesive process is capable of producing pull values higher than current in-process requirements for these products, and that this type of attachment is effective regardless of hole size.

### EXAMPLE 3

Two needle types were used in this example 483947 and 483966, both of which have a 24.8 mil nominal hole diameter. The 483947 needles were finished, and 483966 were unfinished.

Both needle codes were attached to size 1 Vicryl. The exposure time was 2 seconds and the pull testing (n=5) was performed one hour after exposure to the ultra violet light. The adhesive was applied by the syringe injection method.

The finished needle attachment averaged 3.64 lb and failed due to breakdown of adhesive-to-needle bonding, while the unfinished attachments averaged 10.84 lb and failed at the adhesive-to-suture bond.

| Needle Finish | Needle RMC | Pull Averages (n = 5) |
|---|---|---|
| Finished | 483947 | 3.64 lb |
| Unfinished | 483966 | 10.84 lb |

The most likely explanation for the foregoing results is that the polishing process removes the inconsistencies found in the unfinished needle hole which help the adhesive form a mechanical bond. Trace amounts of silicone in the needle hole may also cause poor attachment to the finished needle.

### EXAMPLE 4

Size 3-0 coated Vicryl suture was used for this example.

Unfinished 493966 needles with a 24.8 mil hole were used in this example. To achieve the different hole depths, each needle was manually ground on the hole end until the depth corresponded to the required depth. Samples of these needles were attached with hole depths at nominal depth, 50 mil, 40 mil, and 30 mil. The cure time for each attachment was 3 seconds. Each of the samples were pulled in groups of n=12 immediately after attachment, at 30 minutes from the attachment and 24 hours after attachment. Another n=12 group from each set of samples was put through Ethylene Oxide sterilization (F cycle), then spent four days in “hot room” conditions, and were then pulled.

All four groups showed pull values of less than 1 lb. when pulled immediately after attachment. After 30 minutes, the pull strengths were more widely varied, but all values were significantly higher than the in-process manufacturing requirement of 2.5 lb. After 24 hours, each of the sample sets increased in strength by approximately 18 oz, on average. Following the sterilization process, which took about 3 weeks, each of the samples experienced another increase of approximately 12 oz, on average.

| Hole | Immediate | 30 min. from | 24 hr. from | |
|---|---|---|---|---|
| Depth: | Pull | cure | cure | Sterilized |
| .030″ | 0.72 lb | 3.95 lb | 5.20 lb | 5.68 lb |
| .040″ | 0.68 lb | 4.81 lb | 5.90 lb | 6.87 lb |
| .050″ | 0.80 lb | 5.83 lb | 7.07 lb | 8.05 lb |
| Nominal | 0.88 lb | 6.00 lb | 6.92 lb | 7.68 lb |

The 50 mil hole depth results were indistinguishable from the nominal hole depth of approximately 67 mil. With very few exceptions, all failures after the “immediate” pull were due to adhesive-to-suture bond failures, or suture breaks induced by the rough hole edges left by the grinding process used to achieve the experimental hole depths. This example shows that a reduction in hole depth down to 30 mil still provides enough surface area to produce sufficient pull strength on 3-0 coated Vicryl.

### EXAMPLE 5

Size 2-0 Vicryl was used in this example, as it is the largest suture which was readily available and would fit in each of the reduced holes. The reduced hole needles were 483840, mechanically drilled, chamfered, straight needles (obsolete) which have a nominal hole diameter of 20.2 mil, but were measured in the lab at an average of 20.0 mil. The diameters of the ends of the needles were reduced using a Torrington Swaging unit. The attachment end of each needle was manually inserted into the swager to varying depths from about 0.400″ to 0.420″. This caused hole collapse of anywhere from 0.0015″ to 0.0055″. The resulting holes were then measured with pin gauges and separated into groups with upper hole diameters of 18, 17 and 16 mil (+/−0.2).

Initially, samples of the unmodified 483840s and samples of the same needles reduced to an upper hole diameter of 18 mil (n=12 for both groups) were attached to the size 2-0 coated Vicryl. The adhesive was exposed to low intensity ultra violet light for three seconds, and the samples were pulled after approximately 65 hrs.

Later samples (n=4) of 17 mil and 16 mil reduced holes were attached and pulled according to the same description. The first two samples showed an increase in pull strength from 3.84 lb using the standard hole geometry to 4.94 lb using the 18 mil modified hole geometry. The 17 mil samples showed even more of an increase, to 6.02 lb, as shown in the chart below:

| Top of Hole Reduction | Sample Size | Pull Average |
|---|---|---|
| Nominal | 12 | 61.5 oz |
| .002″ | 12 | 79.0 oz |
| .003″ | 3 | 91.6 oz |
| .004″ | 4 | 25.0 oz |

### EXAMPLE 6

Sizes 5-0, 6-0, 7-0 and 8-0 ProNova* suture were obtained for this test. The needles used in the attachment were 521610 needles, which have a hole diameter of 7.5 mil. This hole is normally swaged with a size 5-0 suture but, consistent with our current method of testing, all sizes in this test were attached using the same hole diameter.

The “syringe injection” method of attachment was not appropriate in this case, as the hole diameter could not accept a syringe needle. Instead, the “suture dip” method was used.

Initially, ten samples using size 5-0 suture were attached and pull tested after approximately 30 minutes. The average of the resulting pull values was 123.5 g (IPQA: 227 g). Examination of the samples showed very low levels of adhesive filling. Further investigation showed inadequate beading of the adhesive on the suture tip due to its low viscosity.

In order to achieve a satisfactory attachment, a small amount of adhesive was left exposed to the ambient atmosphere in an open reservoir for approximately 24 hrs. The resulting effect was a partial curing of the adhesive, and thus an increase in its viscosity. Noticeably higher levels of adhesive beading and filling of the attachment area were achieved.

| | | IPQA Required |
|---|---|---|
| ProNova* Suture Size | Pull Average | Average |
| 5-0 | 207 g | 227 g |
| 6-0 | 112 g | 170 g |
| 7-0 | 84 g | 90 g |
| 8-0 | 95 g | — |

Although adhesive filling of the attachment area was significantly improved using the partially cured adhesive, complete filling was still not achieved. All the low pulls in the test showed poor adhesive filling.

Trailers resulted from the use of the partially cured adhesive, as well. These were fine strands of adhesive which trailed from the attachment area and were a product of the combination of partial curing and suture dipping.

### EXAMPLE 7

This example was conducted to explore briefly the effects of the LOCTITE ® accelerator 7109 and activator 7113 used in conjunction with LOCTITE ® 4302 light curable adhesive. According to LOCTITE ® product information, the 7109 and 7113 products significantly alter the secondary moisture curing properties of 4302 adhesive. Both products induce faster curing, but there is some sacrifice of overall strength with the 7109.

Size 4-0 coated Vicryl suture was used. The suture was attached to 483947 needles, which have a 24.8 mil nominal hole diameter. The accelerators were applied by spraying the fluids toward the hole end of the needle from approximately six inches away. The needles were sprayed three at a time to avoid problems with accelerator evaporation. The suture was then attached using a syringe injection method.

This example compares pull strength when curing is a) unaccelerated or accelerated with b) 7109 or c) 7113 used as an accelerator. Seven samples of each type were attached and pulled after 30 minutes.

The attachments resulting from the use of accelerator 7109 showed the highest pull results, averaging 3.71 lb. For reference, in the Vicryl Time Study, the pull average after 30 minutes was just 2.17, the highest pull average reached (n=12) after 24 hrs was 4.19 lb.

The resulting averages were as follows:

| Using 7109 | Using 7113 | Unaccelerated |
|---|---|---|
| 3.71 lb. | 3.67 lb. | 1.45 lb. |

The use of accelerators caused some amount of difficulty in manual attachment, due to the rapid cure of the adhesive in contact with the accelerators. Also, shallow suture insertions were observed in some of the accelerated samples. These LOCTITE ® accelerators can induce faster cures in the 4302 adhesive. In those cases which showed shallow suture insertions some curing took place in the bottom of the needle hole before the suture could even be inserted.

It should be understood that the embodiments described herein are merely exemplary and that a person skilled in the art may make many variations and modifications without departing from the spirit and scope of the invention as defined in the appended claims. For example, while an exemplary adhesive 18 has been identified as LOCTITE® 4302, other adhesives, such as LOCTITE® 4303, 4304 and 4305, could be employed. All such variations and modifications are intended to be included within the scope of the present invention as defined in the appended claims.

1. An armed suture, comprising:
a needle with a suture hole formed therein;
a suture having an end thereof inserted into said suture hole; and
an adhesive bonding said suture to said needle within said suture hole, said adhesive having a viscosity when uncured permitting said suture to be inserted into said suture hole.

2. The armed suture of claim 1, wherein said adhesive is curable by exposure to electromagnetic radiation.

3. The armed suture of claim 2, wherein said electromagnetic radiation is UV light.

4. The armed suture of claim 3, wherein said adhesive is cyanoacrylate.

5. The armed suture of claim 2, wherein said adhesive is curable by a second curative agent.

6. The armed suture of claim 5, wherein said second curative agent is a compound.

7. The armed suture of claim 5, wherein said second curative agent is water.

8. The armed suture of claim 5, wherein said second curative agent is heat.

9. The armed suture of claim 1, wherein said suture hole has a first diameter D₁ proximate the surface of said needle and a second diameter D₂ distal to said surface of said needle and distal to said first diameter D₁, said second diameter D₂ being greater than said first diameter D₁, such that said adhesive when cured forms a mechanical interlock with said needle hole.

10. The armed suture of claim 9, wherein an end of said needle proximate said needle hole is swaged inwardly.

11. The armed suture of claim 9, wherein said suture hole is formed by laser drilling.

12. The armed suture of claim 1, wherein said suture hole is unpolished.

13. The armed suture of claim 1 wherein said suture hole has at least one groove having an orientation with a component of extension perpendicular to an axial direction of said suture hole.

14. The armed suture of claim 1, wherein said suture is monofilament.

15. A method of making armed sutures, comprising the steps of:
(A) forming a needle hole in an end of a needle;
(B) applying an adhesive between an end of the suture and the needle hole;
(C) inserting a suture into the needle hole
(D) allowing the adhesive to at least partially cure.

16. The method of claim 13, further including a step of metering the adhesive onto the suture hole prior to said step of inserting.

17. The method of claim 16, further comprising a step of secondarily curing the adhesive.

18. The method of claim 17, wherein said step of secondarily curing is performed by exposure to at least one of a chemical curative and heat.

19. The method of claim 18, wherein the chemical curative is applied to the suture hole prior to said step of applying.

20. The method of claim 15, wherein said step of applying includes dipping an end of the suture into the adhesive prior to the step of inserting.
